(19) United States
(12) Patent Application Publication
(10) Pub. No.: US 2026/0354931 A1
(43) Pub. Date: Nov. 12, 2026

(54) Dermaplaning Blade-Wiping Pad System

(71) Applicant: Joshua David Kopinski, Oakdale, MN (US)

(72) Inventor: Joshua David Kopinski, Oakdale, MN (US)

(21) Appl. No.: 19/672,347

(22) Filed: May 8, 2026

Related U.S. Application Data
(60) Provisional application No. 63/802,414, filed on May 8, 2025.

(51) Int. Cl. A61B 90/70; A41D 19/00; A45D 26/00; C09J 5/00; C09J 7/38; C09J 7/40

(52) U.S. Cl. CPC A61B 90/70; A41D 19/0024; A45D 26/0004; C09J 5/00; C09J 7/38; C09J 7/405; C09J 2203/358; C09J 2301/302

(57) ABSTRACT

A more efficient wiping article for wiping dermaplaning skin debris off a dermaplaning blade that frees up both hands of a skin care professional during dermaplaning the skin of a patient. The article is easier to use, provides a better view of the progress of the dermaplaning, and permits easier disposal to the dermaplaning debris. The invention comprises both a system and a method of using the system. The article comprises two elements, a blade-wiping pad releasably adhered to a release liner element.

### FIELD OF THE INVENTION

This invention relates to dermaplaning and the removing and capturing of skin debris during dermaplaning.

### BACKGROUND OF THE INVENTION

There is a need for a better debris wiping system for wiping skin debris comprising at least one of dead skin cells, vellus hair or peach fuzz, sebum and oxidized sebum or blackheads, and makeup residue off of dermaplaning blades used in dermaplaning by skin care professionals.

Existing methods for wiping dermaplaning debris off a dermaplaning blade include for example, esthetician 4 in by 4 in non-woven wipes, cotton towels, and cotton rounds.

Recently, a dermaplaning pad has been introduced that is a thin construction consisting of adhesive-backed felt fabric on a release liner. Wiping dermaplaning debris off a dermaplaning blade during a typical procedure seems difficult, the debris smears on the felt surface, and the felt is hard to separate from the release liner.

There is still a need for a more efficient removal and capture system for wiping dermaplaning debris comprising skin debris off a dermaplaning blade.

### SUMMARY OF THE INVENTION

I have invented a more efficient wiping article for wiping dermaplaning debris off a dermaplaning blade that frees up both hands of a skin care professional during dermaplaning the skin of a patient, is easier to use and more efficiently removes skin debris. The invention is easier to use, provides a better view of the progress of the dermaplaning, and permits easier disposal to the dermaplaning debris. The invention comprises an article that comprises two elements and a method of using the article.

Specifically, the article is a dermaplaning blade-wiping pad system for removing and capturing human skin debris collected on a dermaplaning blade during a dermaplaning procedure by a skin care person. The blade-wiping system comprises a blade-wiping pad and a release liner element.

The blade-wiping pad has a first perimeter, a perimeter edge with a first side and at least a second side and further comprises a contoured sheet element and a pressure sensitive layer.

The contoured sheet element comprises an area, a first thickness, a second perimeter, and a bottom surface layer comprising a second thickness, a first top surface and a first bottom surface. The contoured sheet element also comprises a top surface layer comprising a third thickness, a second top surface having a multitude of contours and a second bottom surface attached to the first top surface of the bottom surface layer to form hollow open chambers configured to remove and capture the skin debris collected on the blade during a dermaplaning procedure. The multitude of contours comprises at least one of a group consisting of (1) at least one collection of multiple parallel ridges, each ridge comprising a first height and at least a first width between multiple parallel crevices, each crevice comprising a first depth, and at least a second width and (2) multiple cavities in the top surface layer with a second depth and a cross-section, wherein the first height is the same as the first depth.

The pressure sensitive adhesive layer comprises a fourth thickness, a third top surface affixed to the first bottom surface of the bottom surface layer of the contoured sheet element with a first force and a third bottom surface configured to releasably adhere with a second force to a back outer surface of a first disposable medical glove during remove the skin debris from the dermaplaning blade with the first force greater than the second force. The pressure sensitive layer also comprises a third perimeter equal to the second perimeter of the contoured sheet element to form the first perimeter of the blade-wiping pad so the entire second bottom surface of the top surface layer is in contact with the third top surface of the pressure sensitive layer.

The release liner element comprises a fourth bottom surface, a fifth thickness, a fourth top surface releasably adhered to the third bottom surface of the pressure sensitive adhesive layer with a third force that is less than the second force, and a fourth perimeter.

The method of using the article comprises seven steps. Specifically, the method is a process of using a dermaplaning blade-wiping pad system for removing and capturing human skin debris from a dermaplaning blade during a dermaplaning procedure by a skin care person. One step to provide at least a first disposable medical glove comprising an inside surface and an outside surface with a back outside surface opposite a palm outside surface and fingers with finger pads on the hand of the skin care person that is to hold the skin taut during dermaplaning. Another step is to provide the blade-wiping pad system as described above. Still another step is to provide the dermaplaning blade to remove skin debris from skin during dermaplaning. Another step is to remove the blade-wiping pad from the release liner element and to attach the blade-wiping pad to the back outside surface of the first disposable medical glove. Still another step is to remove skin debris from skin held taut during dermaplaning and to transfer the skin debris onto the dermaplaning blade without a need to move eyes and attention from the skin that is being dermaplaned. Another step is to wipe the dermaplaning blade on the second top surface of the blade-wiping pad to remove and capture the skin debris from the blade. Still another step is to repeat the last two steps until the dermaplaning procedure is complete.

My invention has numerous advantages over those methods currently used to remove human skin debris during dermaplaning. The dermaplaning blade-wiping pad system of the invention frees both hands for use during dermaplaning. It also quickens the dermaplaning process. In addition, it simplifies sanitary disposal by simply pulling the medical glove attached to the blade-wiping pad with debris off the hand from the wrist, thus encapsulating the blade-wiping pad with debris in the inside-out glove for easy disposal. It also more clearly illustrates the quantity of debris that is being removed by a skin care professional. In addition, it is designed specifically for Dermaplaning treatments and tasks required for dermaplaning blade clearing of skin debris. It also has a special surface texture designed to trap and hold on to skin debris. In addition, it comprises a thin material that is low profile to facilitate easy dermaplaning blade wiping. It also has an adequate size to accommodate a dermaplaning blade.

### BRIEF DESCRIPTION OF THE DRAWINGS

The invention will become more fully understood by the accompanying drawings, wherein like elements are represented by like reference characters, which are given by way of illustration only and thus are not limited to the example embodiments herein.

FIG. 1. is an exploded perspective view looking downward from the side of a first embodiment of the invention with hollow open chambers formed by merging ridges and crevices of two collections of multiple parallel elongated hollow open chambers at 90 degrees from each other on the a second top surface of a top surface layer and a release liner element with a length extending from a first side of the perimeter of the blade-wiping pad of the dermaplaning blade-wiping pad system.

FIG. 2. is the collapsed front side view of the embodiment shown in FIG. 1.

FIG. 3. is the collapsed left side view of the embodiment shown in FIG. 1

FIG. 4. is the collapsed right side view of the embodiment shown in FIG. 1.

FIG. 5. is the collapsed top side view of the embodiment shown in FIG. 1.

FIG. 6. is the collapsed bottom side view of the embodiment shown in FIG. 1.

FIG. 7. is the collapsed back side view of the embodiment shown in FIG. 1.

FIG. 8. is an exploded perspective view looking downward from the side of a second embodiment of the invention with hollow open chambers formed by one collection of multiple parallel elongated hollow open chambers on the a second top surface of a top surface layer and a release liner element with a length extending from a first side and a second side of the perimeter of the blade-wiping pad of the dermaplaning blade-wiping pad system.

FIG. 9. is the collapsed front side view of the embodiment shown in FIG. 8.

FIG. 10. is the collapsed left side view of the embodiment shown in FIG. 8

FIG. 11. is the collapsed right side view of the embodiment shown in FIG. 8.

FIG. 12. is the collapsed top side view of the embodiment shown in FIG. 8.

FIG. 13. is the collapsed bottom side view of the embodiment shown in FIG. 8.

FIG. 14. is the collapsed back side view of the embodiment shown in FIG. 8.

FIG. 15. is an exploded perspective view looking downward from the side of a third embodiment of the invention with hollow open chambers formed multiple cavities on a second top surface of a top surface layer and a release liner element with a length not extending beyond the perimeter edge of the blade-wiping pad of the dermaplaning blade-wiping pad system.

FIG. 16. is the collapsed front side view of the embodiment shown in FIG. 15.

FIG. 17. is the collapsed left side view of the embodiment shown in FIG. 15

FIG. 18. is the collapsed right side view of the embodiment shown in FIG. 15.

FIG. 19. is the collapsed top side view of the embodiment shown in FIG. 15.

FIG. 20. is the collapsed bottom side view of the embodiment shown in FIG. 15.

FIG. 21. is the collapsed back side view of the embodiment shown in FIG. 15.

FIG. 22. is a top view looking downward from the side of the contoured sheet element of the embodiment shown in FIG. 5, with FIG. 22A being a magnified view of a portion of the top surface layer and the bottom surface layer showing several hollow open chambers formed from merging at 90 degrees two collections of parallel ridges and parallel crevices and FIG. 22B being a front view along a line of sight passing through the several hollow open chambers shown in FIG. 22A.

FIG. 23. is a top view looking downward from the side of contoured sheet element of the embodiment shown in FIG. 12, with FIG. 23A being a magnified view of a portion of the top surface layer and the bottom surface layer showing several hollow open chambers, and FIG. 23B being a front view along a line of sight passing through the several hollow open chambers shown in FIG. 23A.

FIG. 24. is a top view looking downward from the side of the contoured sheet element of the embodiment shown in FIG. 15 with FIG. 24A being a magnified view of a portion of the top surface layer and the bottom surface layer showing several cavities and FIG. 24B being a front view along a line of sight passing through the several cavities shown in FIG. 24A.

FIG. 25. is a top view looking downward from the side of fourth embodiment of the invention where contoured sheet element 300 of the embodiment is shown with a fabric having an open weave with cavities as the top surface layer with FIG. 25A being a magnified view of a portion of the top of the top surface layer and the bottom surface layer showing several cavities and FIG. 25B being a front view along a line of sight passing through the several cavities shown in FIG. 25A.

FIG. 26. is a perspective view looking downward from the side of the first embodiment of the invention of the second top surface of the top surface layer having multiple hollow open chambers and a dermaplaning blade being wiped on the second top surface to result in skin debris being deposited upon one side of a ridge of the hollow open chamber.

FIG. 27. is a perspective view looking downward from the side of the first embodiment of the invention receiving skin debris from a dermaplaning blade scraping over blade-wiping pad.

FIG. 28. is a perspective view looking downward from the side of the embodiment shown in FIG. 27 with skin debris after blade-wiping.

FIG. 29. is a perspective view looking downward from the side of a dermatology procedure being performed on a patient by a skin care operator.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF SOME EMBODIMENTS OF THE INVENTION

Dermaplaning is the removal of skin debris from skin with a dermaplaning blade that is scraped over skin that is held taut during the scraping. The blade-wiping pad of the dermaplaning blade-wiping pad system of the invention is a new and innovative device for the skin care professionals that adheres directly to their glove for efficient dermaplaning blade clearing during treatments.

The blade-wiping pad of the invention has five advantages that are particularly beneficial. First, compared to traditional esthetician 4 in by 4 in nonwoven wipes, cotton towels, or cotton rounds, the blade-wiping pads of the invention frees-up both hands. This streamlines the dermaplaning process. Second, since the blade-wiping pad is always in view, dermaplaning blade wiping requires shorter trips to clear the skin debris. This promotes a safer dermaplaning treatment and a more enjoyable experience overall. Third, the blade-wiping pad is made of soft contoured material that collects and holds on to skin debris, allowing uninterrupted use of both hands throughout dermaplaning. Fourth, compared to traditional cotton rounds and wipes, skin debris is highly visible on the color of the blade-wiping pad contrasting such as black when skin debris is a light color. This helps track progress during dermaplaning and highlights how much has been achieved for the client which can lead to increased repeat client visits. Fifth, use of blade-wiping pads allows a clean and sanitary method of disposal of the collected skin debris. Simply remove the glove inside-out to encapsulate the blade-wiping pad and skin debris and dispose in a sanitary manner.

The invention comprises an article that comprises two elements and a method of using the article.

Specifically, the article is a dermaplaning blade-wiping pad system for removing and capturing human skin debris collected on a dermaplaning blade during a dermaplaning procedure by a skin care person. The blade-wiping system comprises a blade-wiping pad and a release liner element.

The blade-wiping pad has a first perimeter, a perimeter edge with a first side and at least a second side and further comprises a contoured sheet element and a pressure sensitive adhesive layer. In some embodiments, the perimeter edge of the blade-wiping pad may have a shape from a group consisting of a square, a rectangle, a triangle, a circle, an oval.

The blade-wiping pad comprises a surface having a color that contrasts with skin debris material removed during the dermaplaning procedure, thereby improving visibility of accumulated skin debris. For example, a dark pad surface can provide enhanced visual contrast for lighter debris while a light pad surface can provide enhanced visual contrast for darker debris, including terminal hair. By increasing contrast between the blade-wiping pad surface and the removed skin debris, the skin care practitioner can more readily observe debris accumulation during use and more effectively monitor procedural progress. In some embodiments, the contoured sheet element may have a color that contrasts with the skin debris collected on a blade during a dermaplaning procedure by a skin care person. In some embodiments, the blade-wiping pad color is a dark color when the skin debris is more distinguishable against a dark color and a light color when the skin debris is more distinguishable against a light color than a dark color.

The contoured sheet element comprises an area, a first thickness, a second perimeter, and a bottom surface layer comprising a second thickness, a first top surface and a first bottom surface. The contoured sheet element also comprises a top surface layer comprising a third thickness, a second top surface having a multitude of contours and a second bottom surface attached to the first top surface of the bottom surface layer to form hollow open chambers configured to remove and capture the skin debris collected on the blade during a dermaplaning procedure. The multitude of contours comprises at least one of a group consisting of (1) at least one collection of multiple parallel ridges, each ridge comprising a first height and at least a first width between multiple parallel crevices, each crevice comprising a first depth, and at least a second width and (2) multiple cavities in the top surface layer with a second depth and a cross-section wherein the first height is the same as the first depth.

The area and first thickness of the contoured sheet element are important for satisfactory use. The area is large enough to accept skin debris from a typical dermaplaning session without being replaced. An area may range from as little as 6 square cm (1 square inch) to over 39 square cm (6 square inches). The first thickness permits a flexible draping of the blade-wiping pad upon the back of a disposable medical glove so that the contoured surface of the contoured sheet element may be used to remove and capture skin debris from a dermaplaning blade. The first thickness is thin and flexible in a draping manner to not interfere with the tactile senses of the gloved hand.

In some embodiments, the at least one collection of multiple parallel ridges between multiple parallel crevices in the top surface layer is at least one collection with all ridges parallel in the same direction and the collection of multiple parallel ridges and multiple parallel crevices is configured to be opposed to the direction of a wiping motion of the blade with skin debris by an angle consisting of more than 15 degrees, more than 30 degrees, more than 45 degrees, more than 60 degrees, and up to 90 degrees. The hollow open chambers are elongated with an end appearing at the second perimeter which may be raised to capture skin debris.

In some embodiments, at least one collection of multiple parallel ridges between multiple parallel crevices in the top surface layer comprises two collections with half of ridges of one collection parallel in one direction with each crevice having the first depth and the second width and half of ridges of the second collection parallel in another direction with each crevice having a third depth and a fourth width and positioned at an angle consisting of more than 15 degrees, more than 30 degrees, more than 45 degrees, more than 60 degrees, and up to 90 degrees to form a crosshatched pattern with similar parallelogram-shaped cavities each with an area formed by the second width and the fourth width and configured to remove and capture skin debris collected on the blade during the dermaplaning procedure when the blade is wiped on the blade-wiping pad.

In some embodiments related to the just mentioned embodiments, the second collection of multiple parallel ridges between multiple parallel crevices in the top surface layer is positioned at an angle consisting of approximately 90 degrees to form a crosshatched pattern with similar square-shaped or rectangular-shaped cavities each with an area formed by the second width and the fourth width and configured to remove and capture skin debris collected on the blade during the dermaplaning procedure when the blade is wiped on the blade-wiping pad. This pattern with a shape or a square or a rectangle makes the direction used to pass the blade over the blade-wiping pad least relevant

In some embodiments, the multiple cavities in the top surface layer form a collection of similar patterns from a group consisting of circles, triangles, squares, rectangles, parallelograms, pentagons, hexagons, and irregular shapes to form cavities with an area configured to remove and capture skin debris collected on the blade during the dermaplaning procedure when the blade is wiped on the blade-wiping pad.

The contoured sheet element that comprises the base surface layer and the top surface layer may be unified or adhered together as separate layers. The base surface layer is generally a thermoplastic film that has a first top surface that forms a base for the multiple hollow open chambers when joined with the second bottom surface of the top surface layer. The top surface layer may be thermoplastic or fabric as long as the multitude of the hollow open chambers are sufficiently sized to remove and capture the skin debris from the dermaplaning blade when it is wiped over the second top surface. When both layers are thermoplastic, the layers are often unified because of the film-like nature of the contoured top surface and the ability of the thermoplastic film to have one side thermoformed into appropriate contours with bases. Suitable thermoplastic films include, for example, polyester, polypropylene, high density polyethylene, low density polyethylene, polyimide, polytetrafluoroethylene, polyvinyl chloride, and nylon. When the top surface layer is a fabric, the layers are separated and adhered together to form the multitude of hollow open chambers. Suitable fabric with multiple cavities having depths and widths sufficient to capture skin debris include, for example, cotton and nylon. However, fabrics with multiple cavities that are either too small or non-existent for satisfactory catching of skin debris during dermaplaning blade cleaning during the dermaplaning process are not suitable and include, for example, felt.

The skin debris comprises at least one following, dead skin cells, vellus hair, sebum & oxidized sebum, and makeup residue. (1) Dead skin cells, also called Corneocytes, are flat, anucleate cells from the stratum corneum. They naturally shed but accumulate on the skin's surface if not exfoliated. Their size has a typical thickness of 0.5 to 1 microns (μm) and a diameter of around 20 to 40 μm. (2) Vellus hair, also called peach fuzz, are fine, light, and short facial hair that dermaplaning can effectively remove. Their size has a diameter of about 10 to 20 μm and a length of about 500 to 2000 μm though that may widely vary. (3) Sebum & oxidized sebum, also called blackheads, can harden to form plugs when the sebaceous secretions oxidize. Size varies depending on accumulation with individual droplets ranging from 1 to 5 μm when in glandular ducts and plug size blackheads ranging from 50 to 500 μm or more. (4) Makeup residue is residual particles from powders or foundations that can be lifted with dermaplaning. Its size varies but power particles can be from 1 to 50 μm.

The hollow open chambers of the contoured sheet element service are configured to remove and capture skin debris similar to that described above without excessive smearing caused by undersized chambers such as those typically on linen. In some embodiments, suitable dimensions comprise the following. The first depth and second depth is at least 0.2 mm (8 mils) and no more than 3.0 mm (120 mils), the first cross-section and second width is at least 0.5 mm (20 mils) and no more than 4.0 mm (160 mils), and the second thickness is at least 0.23 mm (9 mils) and no more than 1.5 mm (60 mils) to permit satisfactory removal and capture of the skin debris collected on the blade during the dermaplaning procedure. Other dimensions are also possible as long as satisfactory removal and capture of the skin debris collected on the blade occurs during the dermaplaning procedure.

The pressure sensitive adhesive layer comprises a fourth thickness, a third top surface affixed to the first bottom surface of the bottom surface layer of the contoured sheet element with a first force and a third bottom surface configured to releasably adhere with a second force to a back outer surface of a first disposable medical glove during remove the skin debris from the dermaplaning blade with the first force greater than the second force. This prevents potential separation of the contoured sheet element from the pressure sensitive adhesive layer when the multiple hollow open chambers are filled with skin debris. The pressure sensitive layer also comprises a third perimeter equal to the second perimeter of the contoured sheet element to form the first perimeter of the blade-wiping pad so the entire second bottom surface of the top surface layer is in contact with the third top surface of the pressure sensitive layer.

The pressure-sensitive adhesive layer is from an adhesive that is configured to releasably adhere to the back of a first disposable medical glove. The pressure-sensitive adhesive may be, for example, of a group consisting of acrylic, silicone, rubber, or silicone-rubber. The pressure-sensitive adhesive is configured to release from a release liner element when a blade-wiping pad is grasped with a hand in a first disposable medical glove, transferred to a second disposable medical glove, and releasably adhered to the back of the first disposable medical glove.

While the pressure-sensitive adhesive is releasably adhered to both the back of the glove as well as the fingers of the disposable medical glove to permit removal of the blade-wiping pad from the fingers during transfer from the release liner element to the back of the disposable medical glove, it is preferable not to remove the blade-wiping pad from the back of the disposable medical glove when skin debris is wiped upon it. Such removal may also dislodge skin debris from the blade-wiping pad to scatter about a work area where dermaplaning is occurring or has occurred.

In some embodiments, the second force is less than the first force and the second force is configured to be more than a fourth force holding the third bottom surface of the pressure sensitive adhesive layer to the disposable medical glove digit pads during transfer of the blade-wiping pad from the release liner element to the back outside surface of the disposable medical glove. This facilitates wearing both disposable medical gloves during a dermaplaning procedure while removing the blade-wiping pad from the release liner element and placing it on the back the first disposable medical glove.

The release liner element comprises a fourth bottom surface, a fifth thickness, a fourth top surface releasably adhered to the third bottom surface of the pressure sensitive adhesive layer with a third force that is less than the second force, and a fourth perimeter.

The fourth perimeter of the release liner element may or may not extend outside the first perimeter of the blade-wiping pad. In some embodiments, the fourth perimeter of the release liner element does not extend outside the first perimeter of the blade-wiping pad. This may result in more blade-wiping pads on a sheet of release liner. In some embodiments, the fourth perimeter of the release liner element may extend outside the first perimeter of the blade-wiping pad by a length on at least the first side of the perimeter edge of the blade-wiping pad. This may result in fewer blade-wiping pads on a sheet of release liner, but each will be easier to peel off the liner in one direction. In some embodiments, the fourth perimeter of the release liner element may extend outside the first perimeter of the blade-wiping pad by a length on more than the first side of the perimeter edge of the blade-wiping pad up to the entire first perimeter. This may result in fewest blade-wiping pads on a sheet of release liner, but each will be easier to peel off the liner from multiple directions.

The release liner element may extend beyond the blade-wiping pad on one side of the perimeter of the blade-wiping pad or more than one side, up to the entire first perimeter edge. In some embodiments, where the length between the first perimeter of the blade-wiping pad and the fourth perimeter of the release liner element is on the first side of the first perimeter edge of the blade-wiping pad, the length is from a group consisting of at least 1 mm (40 mils), at least 2 mm (80 mils), at least 3 mm (120 mils), at least 4 mm (160 mils), and at least 5 mm (200 mils) to permit easy removal of the blade-wiping pad from the release liner element. In some embodiments, where the length between the first perimeter of the blade-wiping pad and the fourth perimeter of the release liner element is on the first side and at least the second side of the first perimeter edge of the blade-wiping pad, the length may vary where a length exists from the entire perimeter edge is from a group consisting of at least 1 mm (40 mils), at least 2 mm (80 mils), at least 3 mm (120 mils), at least 4 mm (160 mils), and at least 5 mm (200 mils) for easy removal from any part of the perimeter edge.

In some embodiments, the pressure sensitive adhesive is configured to release from the release liner element when a pad is grasped with a hand in a first glove, transferred to a second glove, and releasably adhered to the back of the first glove.

Pressure-sensitive adhesives able to provide the forces described above are well known and include, for example, coming from a group consisting of acrylic, silicone, rubber, silicone-rubber, and modified epoxy.

In some embodiments, the release liner element is configured with flexibility to permit bending the release liner element fourth bottom surface back upon itself under the blade-wiping pad where the fourth top surface of the release liner element touches the first perimeter of the blade-wiping pad on a perimeter edge side where the length of release liner element exists and wherein the length of release liner element is configured to permit a person with fingers to grasp the edge of the blade-wiping pad. The blade-wiping pad is then removed from the top surface of the release liner element and placed on the back outside surface of the first disposable medical glove to be adhered to the back outside surface of the first disposable medical glove. In some embodiments of the above, the fingers used to grasp the edge of the blade-wiping pad may be within finger pads of fingers of a second disposable medical glove. In some embodiments, the fifth thickness of the release liner element is at least 0.1 mm (4 mils) and no more than 0.5 mm (20 mils). In some embodiments, the fifth thickness may be more or less depending on the stiffness of the material used to make the release liner element

A blade-wiping pad system may have various shapes and dimensions. A representative description follows for an embodiment having two collections of multiple parallel ridges each with same ridge height, ridge width, and crevice width, merged at 90 degrees to form square hollow open chambers. The blade-wiping pad has a length of 52 mm (2 in), a length of 34 mm (1.4 in), and a thickness of 0.4 mm (16 mils). The contours have a ridge height of 0.3 mm (12 mils), a ridge width of 1.0 mm (40 mils), chamber crevice or cavity cross section of 1.0 mm (40 mils) by 1.0 mm (40 mils). The pressure sensitive adhesive has a thickness of 0.05 mm (2 mils). The release liner element has a length of 65 mm (2.5 in), a width of 45 mm (1.8 in) and a thickness of 0.1 mm (4 mils). The contoured sheet element is black for contrast with skin debris.

The dermaplaning blade-wiping system may come in various forms for use by skin care professionals. The blade-wiping pad on the release liner element may be in a roll for easy dispensing. They may also be in the form of a stack of pads for easy storage.

The disposable medical gloves typically used in dermaplaning procedures are disposable medical exam gloves available in the medical and cosmetic arts. They are typically vinyl-free. One brand, for example, is Salon Care Nitrile Gloves sold by Sally Beauty Supply.

The dermaplaning blade-wiping system may be better understood by viewing various figures that are meant to represent some embodiments and not be limiting.

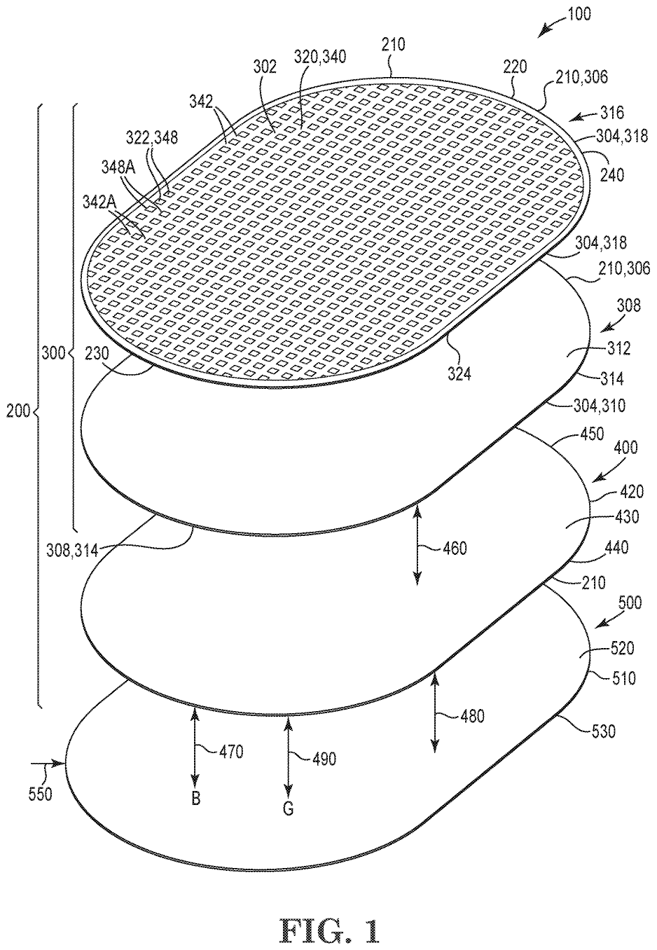

FIG. 1. is an exploded perspective view looking downward from the side of a first embodiment of the invention with hollow open chambers formed by merging ridges and crevices of two collections of multiple parallel elongated hollow open chambers at 90 degrees from each other on a second top surface of a top surface layer and a release liner element with a length extending from a first side of the perimeter of the blade-wiping pad of the dermaplaning blade-wiping pad system. A dermaplaning blade-wiping pad system (100) is shown with individual layers separated. Blade-Wiping Pad (BWP) (200) is shown with a BWP first perimeter (210), a BWP perimeter edge (220), a BWP perimeter edge first side (230), and a BWP perimeter edge at least second side (240). Contoured Sheet Element (CSE) (300) is shown with a CSE area (302), CSE first thickness (304), a CSE second perimeter (306), and a CSE Bottom Surface Layer (CSE BSL) (308) with a CSE BSL second thickness (310), a CSE BSL first top surface (312), and a CSE BSL First Bottom Surface CSE TSL FBS) (314). CSE 300 is also shown with a CSE Top Surface Layer (CSE TSL) (316) with a CSE TSL third thickness (318), a CSE TSL Second Top Surface (320, a CSE TSL STS multitude of contours (322), and a CSE TSL second bottom surface (324). A CSE TSL also is shown with a Multitude of Hollow Open Chambers (MHOC) (340) formed by the merging of one collection of parallel ridges and crevices with a MHOC Collection of Multiple Parallel Ridges (MCOC CMPR) (342), and a MHOC Collection of Multiple Parallel Crevices (MCOC CMPC) (348), with a second collection of parallel ridges and crevices oriented at 90 degrees from the first and shown as a MHOC 2Collection of Multiple Parallel Ridges (MCOC CMPR) (342A), and a MHOC 2Collection of Multiple Parallel Crevices (MCOC CMPC) (348A). A Pressure Sensitive Adhesive Layer (PSAL) (400) is shown with a PSAL fourth thickness (420), a PSAL Third Top Surface (430), a PSAL Third Bottom Surface (PSAL TBS) (440), and a PSAL third perimeter (450). Also shown is a Release Liner Element (RLE) (500) with a RLE fifth thickness (510), a RLE Fourth Top Surface (RLE FTS) (520) a RLE fourth bottom surface (530), and a RLE length (540). Also indicated is a first force (460) between CSE FBS and PSAL TTS, a second force (470) between PSAL TBS and Disposable Medical Glove First Hand (DMG FH) back outside surface (B) that is not shown), a third force (480) between PSAL TBS and RLE FTS and a fourth force (490) between PSAL TBS and DMG FH digit pads G that is not shown.

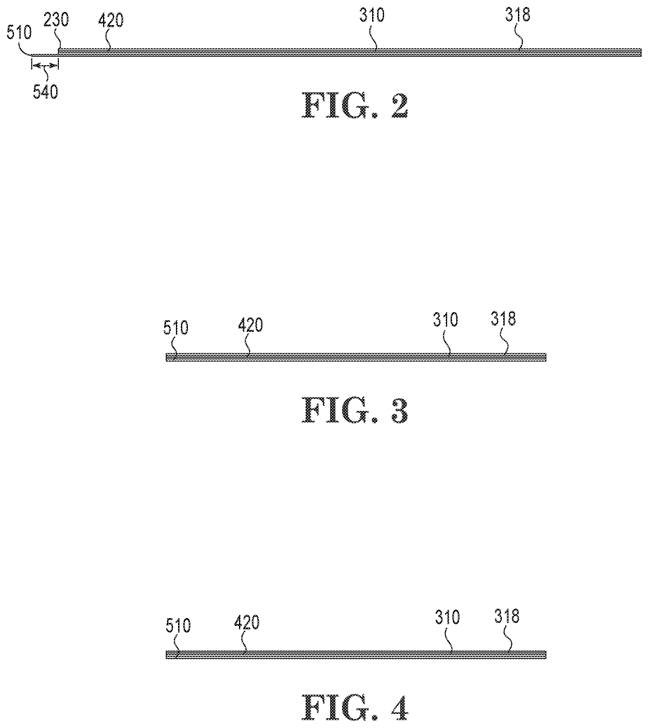

FIG. 2. is the collapsed front side view of the embodiment shown in FIG. 1. Shown are contoured sheet element top surface layer third thickness 318, contoured sheet element bottom surface layer second thickness 310, pressure sensitive adhesive layer fourth thickness 420, release liner fifth thickness 510 and release liner element length 540 that extends beyond blade-wiping pad perimeter first edge 230.

FIG. 3. is the collapsed left side view of the embodiment shown in FIG. 1. Shown are contoured sheet element top surface layer third thickness 318, contoured sheet element bottom surface layer second thickness 310, pressure sensitive adhesive layer fourth thickness 420, and release liner fifth thickness 510.

FIG. 4. is the collapsed right side view of the embodiment shown in FIG. 1. Shown are contoured sheet element top surface layer third thickness 318, contoured sheet element bottom surface layer second thickness 310, pressure sensitive adhesive layer fourth thickness 420, and release liner fifth thickness 510.

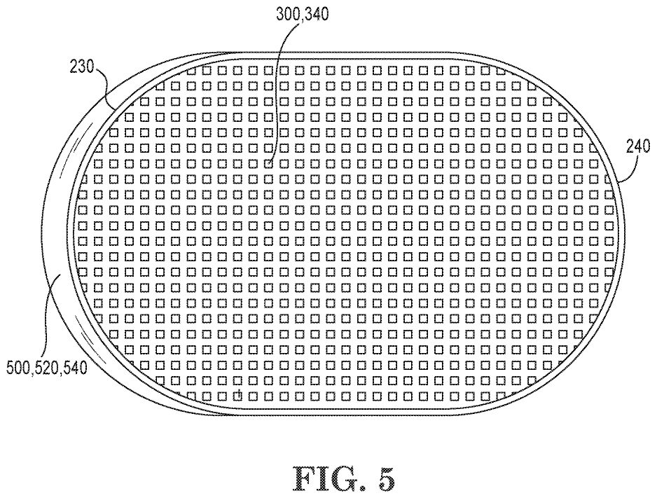

FIG. 5. is the collapsed top side view of the embodiment shown in FIG. 1. Shown are contoured sheet element 300, multitude of hollow open chambers 340, release liner element 500, release liner element fourth top surface 530, and release liner element length 540 extending beyond blade-wiping pad perimeter first edge 230.

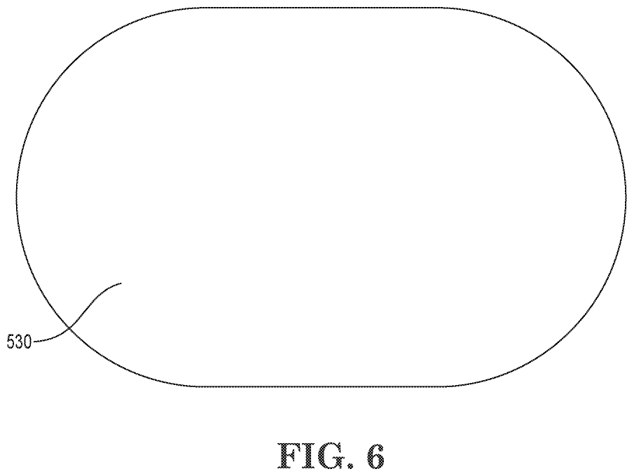

FIG. 6. is the collapsed bottom side view of the embodiment shown in FIG. 1. Shown is release liner element fourth bottom surface 530.

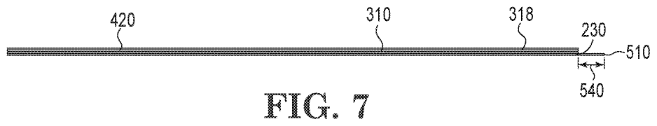

FIG. 7. is the collapsed back side view of the embodiment shown in FIG. 1. Shown are contoured sheet element top surface layer third thickness 318, contoured sheet element bottom surface layer second thickness 310, pressure sensitive adhesive layer fourth thickness 420, release liner fifth thickness 510 and release liner element length 540 that extends beyond blade-wiping pad perimeter first edge 230.

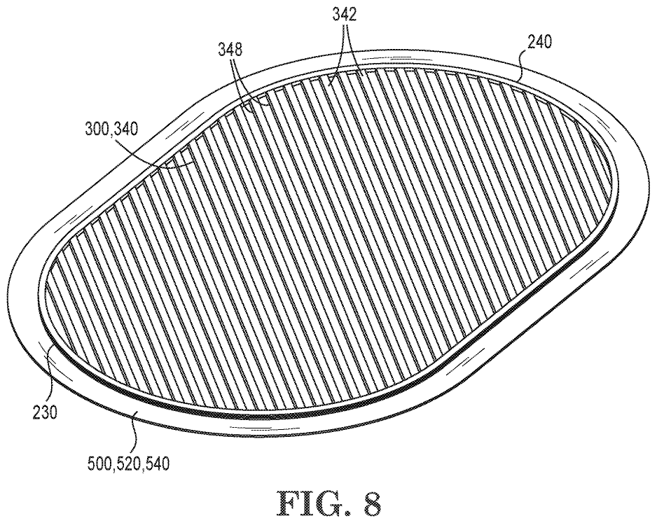

FIG. 8. is an exploded perspective view looking downward from the side of a second embodiment of the invention with hollow open chambers formed by one collection of multiple parallel elongated hollow open chambers with ends at blade-wiping pad first perimeter on the second top surface of the top surface layer and release liner element with length extending from perimeter of blade-wiping pad of dermaplaning blade-wiping pad system. The second embodiment is similar to that of FIG. 1 except only one Collection of Multiple Parallel Ridges 342 and one Collection of Multiple Parallel Crevices 348 is shown on contoured sheet element 300 and multitude of hollow open chambers 340, and release liner element 500 is shown with release liner element fourth top surface 530 and release liner element length 540 extending beyond blade-wiping perimeter edge first side 230 and blade-wiping pad at least second side 240.

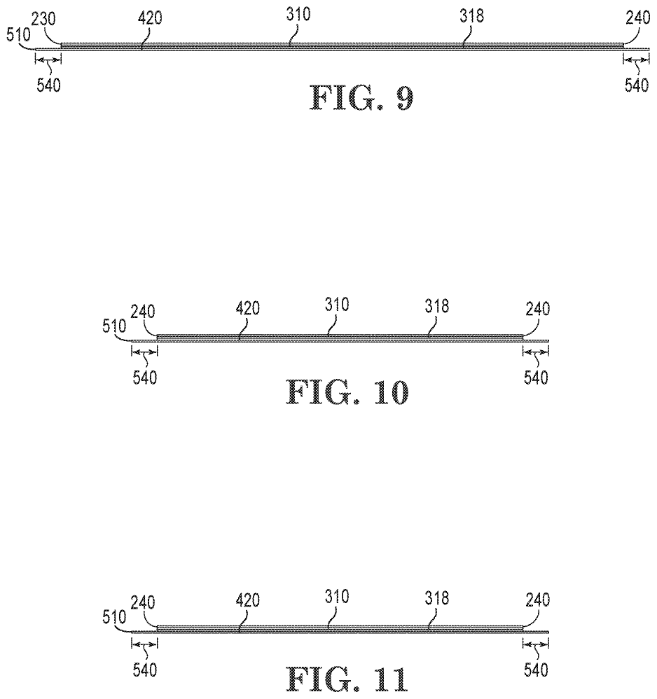

FIG. 9. is the collapsed front side view of the embodiment shown in FIG. 8. Shown are contoured sheet element top surface layer third thickness 318, contoured sheet element bottom surface layer second thickness 310, pressure sensitive adhesive layer fourth thickness 420, release liner fifth thickness 510 and release liner element length 540 that extends beyond blade-wiping pad perimeter first edge 230 and blade-wiping pad perimeter edge at least second side 240.

FIG. 10. is the collapsed left side view of the embodiment shown in FIG. 8. Shown are contoured sheet element top surface layer third thickness 318, contoured sheet element bottom surface layer second thickness 310, pressure sensitive adhesive layer fourth thickness 420, release liner fifth thickness 510 and release liner element length 540 that extends beyond blade-wiping pad perimeter first edge 230 and blade-wiping pad perimeter edge at least second side 240.

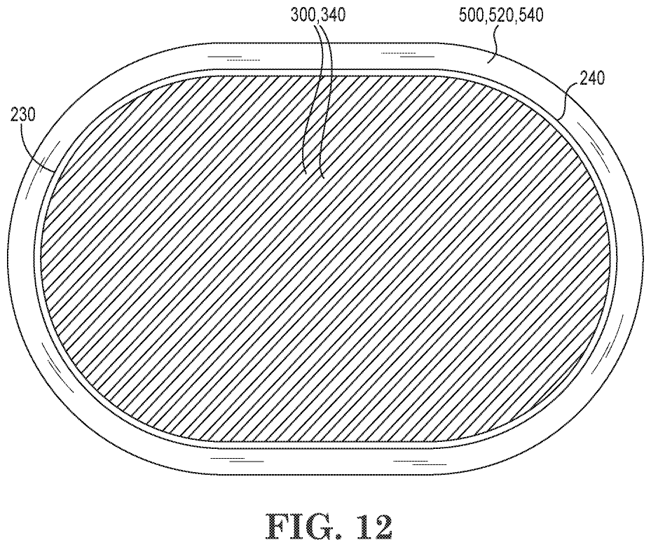

FIG. 11. is the collapsed right side view of the embodiment shown in FIG. 8. Shown are contoured sheet element top surface layer third thickness 318, contoured sheet element bottom surface layer second thickness 310, pressure sensitive adhesive layer fourth thickness 420, release liner fifth thickness 510 and release liner element length 540 that extends beyond blade-wiping pad perimeter first edge 230 and blade-wiping pad perimeter edge at least second side 240.

FIG. 12. is the collapsed top side view of the embodiment shown in FIG. 8. Shown are contoured surface element 300 with top surface layer multitude of hollow open chambers 340 from one collection of multiple ridges and crevice and release liner element 500 with release liner element fourth top surface 530 and release liner element length 540 extending beyond blade-wiping perimeter edge first side 230 and blade-wiping pad at least second side 240

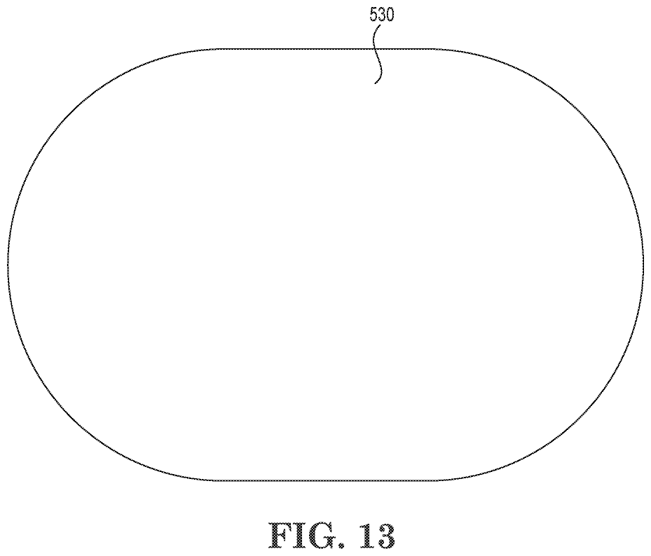

FIG. 13. is the collapsed bottom side view of the embodiment shown in FIG. 8. Shown is release liner element fourth bottom surface 530.

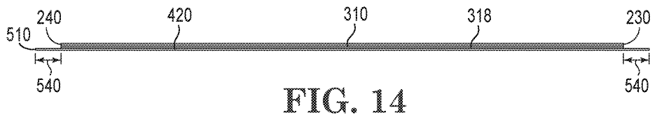

FIG. 14. is the collapsed back side view of the embodiment shown in FIG. 8. Shown are contoured sheet element top surface layer third thickness 318, contoured sheet element bottom surface layer second thickness 310, pressure sensitive adhesive layer fourth thickness 420, release liner fifth thickness 510 and release liner element length 540 that extends beyond blade-wiping pad perimeter first edge 230 and blade-wiping pad perimeter edge at least second side 240.

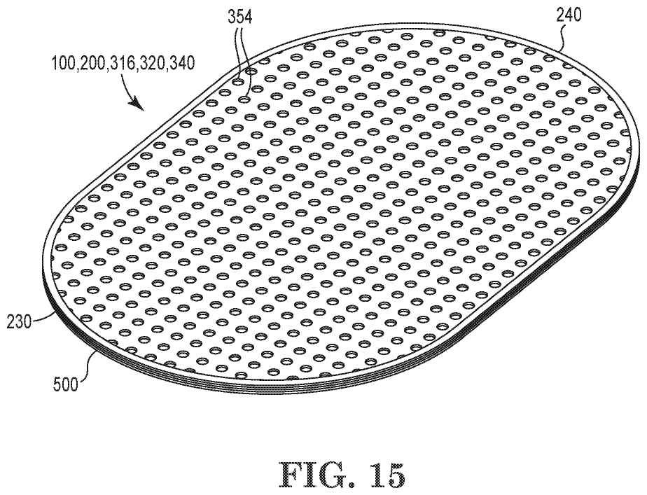

FIG. 15. is a collapsed perspective view looking downward from the side of a third embodiment of the invention with multitude of hollow open chambers formed as multiple cavities in the form of cylinders on second top surface of top surface layer and release liner element with no length extending beyond blade-wiping pad perimeter edge of the blade-wiping pad of the dermaplaning blade-wiping pad system. Shown is contoured sheet element 300 with multitude of hollow open chambers 340 formed as multiple cavities (354) in the form of cylinders on second top surface 320 of top surface layer 316 and release liner element 500 with no length extending beyond blade-wiping pad perimeter edge 220 of the blade-wiping pad 200 of the dermaplaning blade-wiping pad system 100.

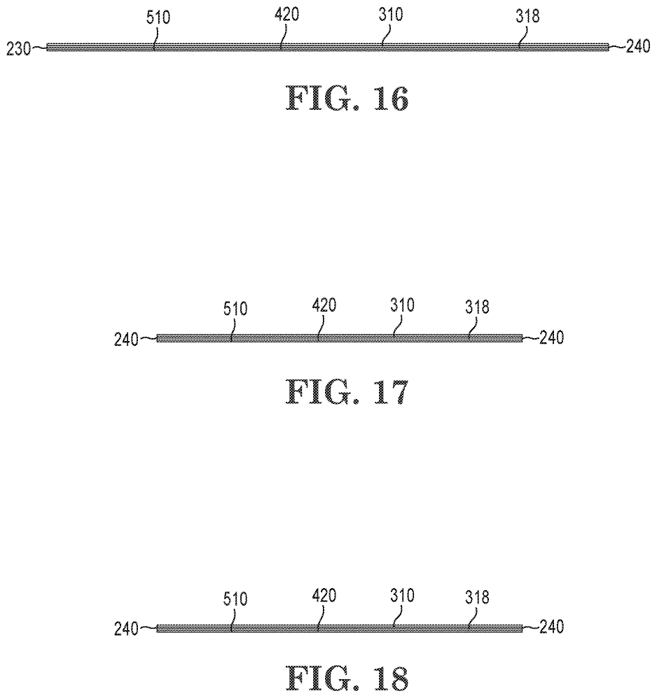

FIG. 16. is the collapsed front side view of the embodiment shown in FIG. 15. Shown are contoured sheet element top surface layer third thickness 318, contoured sheet element bottom surface layer second thickness 310, pressure sensitive adhesive layer fourth thickness 420, release liner fifth thickness 510 and no length 540 that extends beyond blade-wiping pad perimeter first edge 230.

FIG. 17. is the collapsed left side view of the embodiment shown in FIG. 15. Shown are contoured sheet element top surface layer third thickness 318, contoured sheet element bottom surface layer second thickness 310, pressure sensitive adhesive layer fourth thickness 420, and release liner fifth thickness 510.

FIG. 18. is the collapsed right side view of the embodiment shown in FIG. 15. Shown are contoured sheet element top surface layer third thickness 318, contoured sheet element bottom surface layer second thickness 310, pressure sensitive adhesive layer fourth thickness 420, and release liner fifth thickness 510.

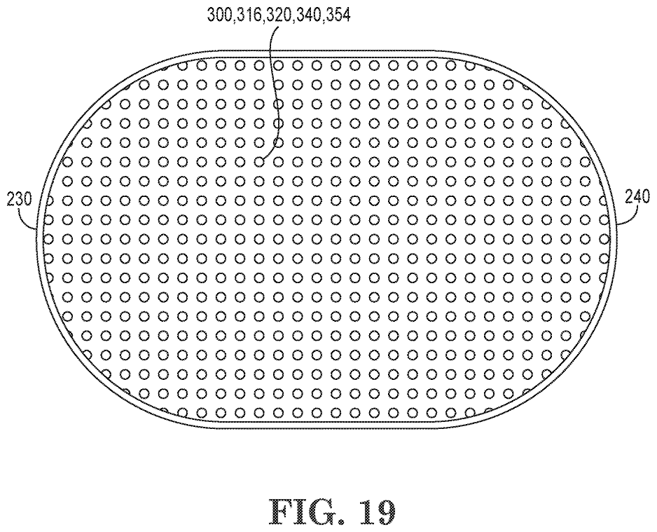

FIG. 19 is the collapsed top side view of the embodiment shown in FIG. 15. Shown is contoured sheet element 300 with multitude of hollow open chambers 340 formed as multiple cavities 354 in the form of circles on second top surface 320 of top surface layer 316 and release liner element 500.

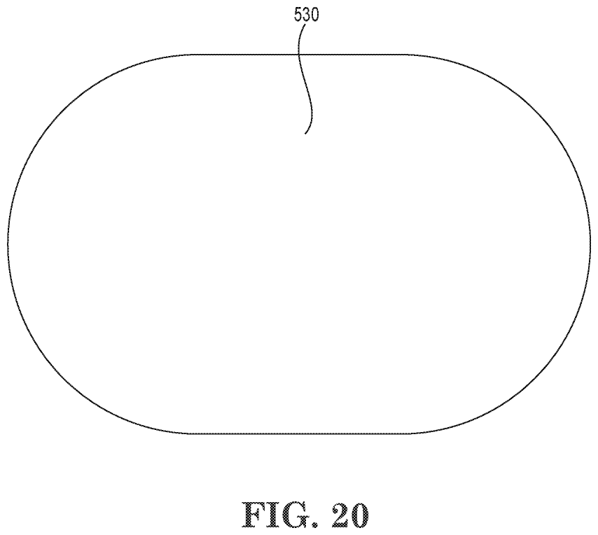

FIG. 20. is the collapsed bottom side view of the embodiment shown in FIG. 15. Shown is release liner element fourth bottom surface 530.

FIG. 21. is the collapsed back side view of the embodiment shown in FIG. 15. Shown is dermaplaning blade-wiping pad system 100 with blade-wiping pad 200 on release liner element 500. Also shown is contoured sheet element 300, top surface layer 316, second top surface 320, and multitude of hollow open chambers 340.

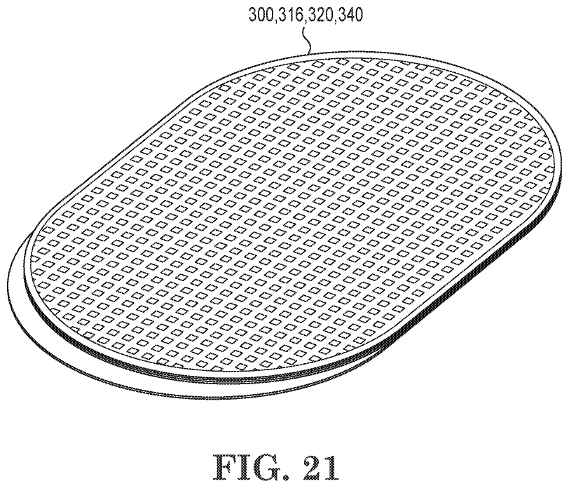

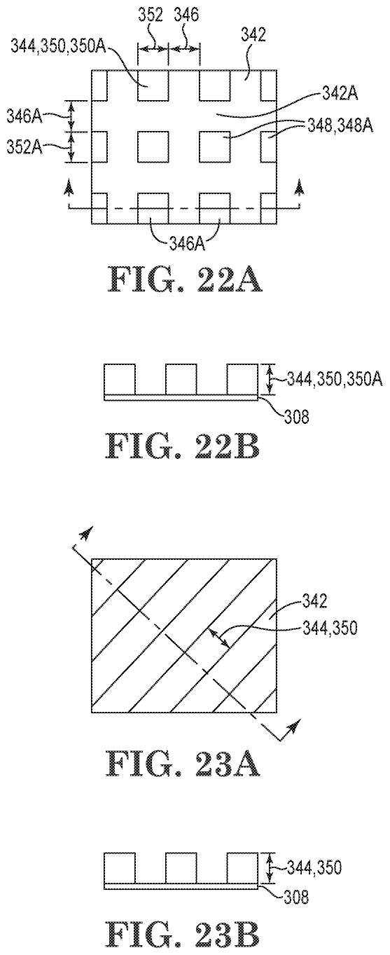

FIG. 22 is a top view looking downward from the side of the contoured sheet element of the embodiment shown in FIG. 5, with FIG. 22A being a magnified view of a portion of the top surface layer and the bottom surface layer showing several hollow open chambers and FIG. 22B being a front view along a line of sight passing through the several hollow open chambers shown in FIG. 22A. In FIG. 22A one collection is shown of ridges having a first width (346) and crevices having a first depth (350) and a second width (352) and a second coaction of ridges having a third width (346) and crevices having a third depth (350A) and a fourth width (352A). In FIG. 22B several hollow open chambers are shown having first height 344 and first depths 350 from one collection and a third depth (350A) from the second collection. Also shown is a portion of bottom surface layer 308.

FIG. 23. is a top view looking downward from the side of contoured sheet element 300 of the embodiment shown in FIG. 12, with FIG. 23A being a magnified view of a portion of the top surface layer and the bottom surface layer showing several hollow open chambers, and FIG. 23B being a front view along a line of sight passing through the several hollow open chambers shown in FIG. 23A. In FIG. 23A, contoured sheet element 300 is shown with top surface layer 316 with multitude of open hollow chambers 322 formed by one elongated collection of multiple parallel ridges having a first height of 344 and multiple parallel crevices having first depth 350 and second width 352. In FIG. 23B, the side view of the contoured sheet element of FIG. 23A shows the several chambers having a ridge with a first height 344 and crevice with a depth of 350. Also shown is a portion of base surface layer 308.

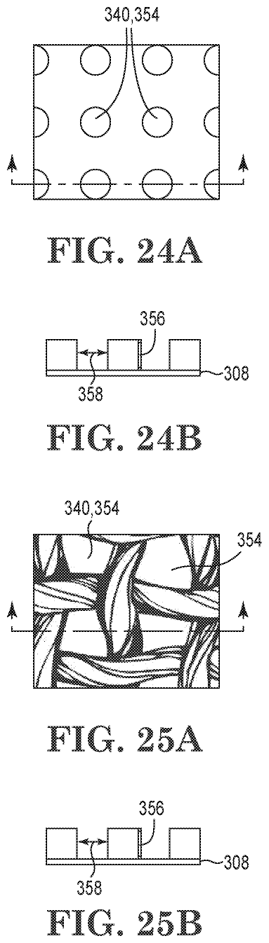

FIG. 24. is a top view looking downward from the side of the contoured sheet element of the embodiment shown in FIG. 15 with FIG. 24A being a magnified view of a portion of the top surface layer and the bottom surface layer showing several cavities and FIG. 24B being a front view along a line passing through the several cavities shown in FIG. 24A. In FIG. 24A, multitude of hollow open chambers 340 are shown formed as multiple cavities 354 in the shape of circles having a cross-section (358). In FIG. 24B, multitude of cavities are shown having second depth 356 and cross-section 358. Also shown is a portion of base surface layer 308.

FIG. 25. is a top view looking downward from the side of fourth embodiment of the invention where contoured sheet element 300 of the embodiment is shown with a fabric having an open weave with cavities as the top surface layer with FIG. 25A being a magnified view of a portion of the top of the top surface layer and the bottom surface layer showing several cavities and FIG. 25B being a front view along a line passing through the several cavities shown in FIG. 25A. In FIG. 25A, multitude of hollow open chambers 340 are shown formed as multiple cavities 354 in the open weave of the fabric having cross-section 358. In FIG. 25B, multiple cavities are shown having second depth 356 and cross-section 358. Also shown is a portion of base surface layer 308.

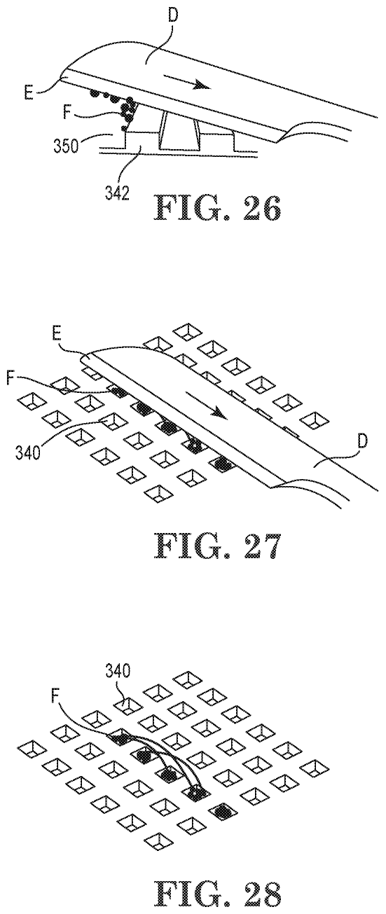

FIG. 26. is a close-up perspective view looking downward from the side of the first embodiment of the invention of a hollow open chamber being used to remove skin debris from a dermaplaning blade. Shown is skin debris (F) falling into a crevice depth first 350 as it is scraped from a dermaplaning blade (E) with skin debris against a ridge 342.

FIG. 27. is a perspective view looking downward from the side of the first embodiment of the invention receiving skin debris from a dermaplaning blade scraping over blade-wiping pad. Shown are with a multitude of hollow open chambers 340 with skin debris F being deposited into some of the chambers as a dermaplaning blade edge is scraped over the chambers.

FIG. 28. is a perspective view looking downward from the side of the embodiment shown in FIG. 27 with skin debris after blade-wiping. Shown is skin debris F in several hollow open chambers 340 with others still empty.

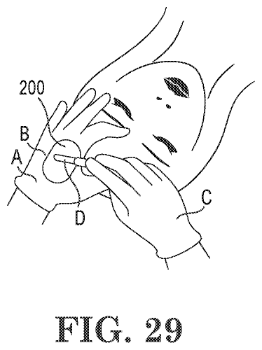

FIG. 29. is a perspective view looking downward from the side of a dermatology procedure being performed on a patient by a skin care operator. Shown is a blade-wiping pad 200 adhered to the back (B) of a first surgical glove (A) worn by one hand of the skin care operator while the fingers of the skin care operator stretch the patient's skin and the blade (E) is held by the hand of the skin care operator in a second surgical glove (C).

The method is a process of using a dermaplaning blade-wiping pad system for removing and capturing human skin debris from a dermaplaning blade during a dermaplaning procedure by a skin care person. The method comprises seven steps.

One step to provide at least a first disposable medical glove comprising an inside surface and an outside surface with a back outside surface opposite a palm outside surface and fingers with finger pads on the hand of the skin care person that is to hold the skin taut during dermaplaning.

Another step is to provide the blade-wiping pad system as described above.

Still another step is to provide the dermaplaning blade to remove skin debris from skin during dermaplaning.

Another step is to remove the blade-wiping pad from the release liner element and to attach the blade-wiping pad to the back outside surface of the first disposable medical glove.

Still another step is to remove skin debris from skin held taut during dermaplaning and to transfer the skin debris onto the dermaplaning blade without a need to move eyes and attention from the skin that is being dermaplaned.

Another step is to wipe the dermaplaning blade on the second top surface of the blade-wiping pad to remove and capture the skin debris from the blade.

Still another step is to repeat the last two steps until the dermaplaning procedure is complete.

In some embodiments, the method further steps two steps.

One step is to remove the first disposable medical glove with the blade-wiping pad with skin debris by grasping the wrist area and pulling the first disposable medical glove off the hand while allowing the first disposable medical glove to become inside out and thereby confining the blade-wiping pad with skin debris in a sanitary manner. Another step is to dispose of the inside out first disposable medical glove containing the blade-wiping pad with skin debris.

In some embodiments of the method, the blade-wiping pad may be removed from the release liner element with a second disposable medical glove worn by the skin care person on the dominant hand.

The contoured sheet element is made from a material that is flexible and has multiple hollow chambers configured to remove and capture the skin debris collected on the blade during a dermaplaning procedure. Flexibility allows the blade-wiping pad to drape over the back of the disposable medical glove in a comfortable manner to the skin care professional. The chambers are formed by combining a contoured surface layer having a multitude of contours layer and a base layer having a continuous form line a film. The multitude of contours comprises at least one of a group consisting of (1) at least one collection of multiple parallel ridges, each ridge comprising a first height and at least a first width between multiple parallel crevices, each crevice comprising a first depth, and at least a second width and (2) multiple cavities in the top surface layer with a second depth and a cross-section wherein the first height is the same as the first depth. Where the contours are from (1), the contoured surface layer may be made from plastic that is heat-formed into a contoured top surface layer and a continuous base layer that is usually unified. When the contours are from (2), the contoured surface layer may be made from plastic that is heat-formed into a contoured surface layer and a continuous base layer that is usually unified, or may be a fabric that is adhered to the base by such methods as heat welding or adhesive to a plastic base. Pressure-sensitive adhesives and release liners are well known and embodiments selected for satisfactory use with the dermaplaning blade-wiping pad system of the invention may be easily determined once the parameters desired for the invention are determined.

List of Elements

| A | Disposable Medical Glove first hand |
|---|---|
| B | DMG FH back outside surface |
| C | DMG Second Hand |
| D | Dermaplaning blade DB |
| E | DB edge |
| F | Skin debris |
| G | DMC SH digit pads |
| 100 | D blade-wiping pad system |
| 200 | Blade-Wiping Pad |
| 210 | BWP first perimeter |
| 220 | BWP perimeter edge |
| 230 | BWP perimeter edge first side |
| 240 | BWP perimeter edge at least second side |
| 300 | Contoured Sheet Element |
| 302 | CSE area |
| 304 | CSE first thickness |
| 306 | CSE second perimeter |
| 308 | CSE Bottom Surface Layer |
| 310 | CSE BSL second thickness |
| 312 | CSE BSL first top surface |
| 314 | CSE BSL First Bottom Surface |
| 316 | CSE Top Surface Layer |
| 318 | CSE TSL third thickness |
| 320 | CSE TSL Second Top Surface |
| 322 | CSE TSL STS multitude of contours |
| 324 | CSE TSL second bottom surface |
| 340 | Multitude of Hollow Open Chambers |
| 342 | MHOC Collection Multiple Parallel Ridges |
| 344 | MHOC CMPR first height |
| 346 | MHOC CMPR first width |
| 348 | MHOC Collection Multiple Parallel Crevices |
| 350 | MHOC CMPC first depth |
| 352 | MHOC CMPC second width |
| 342A | MHOC 2Collection Multiple Parallel Ridges |
| 346A | MHOC 2CMPR third width |
| 348A | MHOC Collection Multiple Parallel Crevices |
| 350A | MHOC 2CMPC third depth |
| 352A | MHOC 2CMPC fourth width |
| 354 | MHOC Multiple Cavities |
| 356 | MHOC MC second depth |
| 358 | MHOC MC cross-section |
| 400 | Pressure Sensitive Adhesive Layer |
| 420 | PSAL fourth thickness |
| 430 | PSAL Third Top Surface |
| 440 | PSAL Third Bottom Surface |
| 450 | PSAL third perimeter |
| 460 | First force b/t CSE BSL FBS & PSAL TTS |
| 470 | Second force PSAL TBS & B |
| 480 | Third force b/t PSAL TBS & RLE FTS |
| 490 | Fourth force b/t PSAL TBS & G |
| 500 | Release Liner Element |
| 510 | RLE fifth thickness |
| 520 | RLE Fourth Top Surface |
| 530 | RLE fourth bottom surface |
| 540 | RLE length |

I claim:

1. A dermaplaning blade-wiping pad system for removing and capturing human skin debris collected on a blade during a dermaplaning procedure by a skin care person comprising,
a blade-wiping pad having a first perimeter, a perimeter edge with a first side and at least a second side, and comprising
a contoured sheet element comprising an area, a first thickness, a second perimeter, a bottom surface layer comprising a second thickness, a first top surface and a first bottom surface, and a top surface layer comprising a third thickness, a second top surface having a multitude of contours and a second bottom surface attached to the first top surface of the bottom surface layer to form hollow open chambers configured to remove and capture the skin debris collected on the blade during a dermaplaning procedure wherein the multitude of contours comprises at least one of a group consisting of (1) at least one collection of multiple parallel ridges, each ridge comprising a first height and a first width between multiple parallel crevices, each crevice comprising a first depth, and at least a second width and (2) multiple cavities in the top surface layer with a second depth and a cross-section, wherein the first height is the same as the first depth; and
a pressure sensitive adhesive layer comprising a fourth thickness, a third top surface affixed to the first bottom surface of the bottom surface layer of the contoured sheet element with a first force, a third bottom surface configured to releasably adhere with a second force to a back outer surface of a first disposable medical glove during the dermaplaning procedure with the first force greater than the second force, and a third perimeter equal to the second perimeter of the contoured sheet element to form the first perimeter of the blade-wiping pad so the entire surface of the first bottom of the contoured sheet element is in contact with the second top surface of the pressure sensitive layer pad so the entire second bottom surface of the top surface layer is in contact with the third top surface of the pressure sensitive layer; and
a release liner element comprising a fourth bottom surface, a fifth thickness, a fourth top surface releasably adhered to the third bottom surface of the pressure sensitive adhesive layer with a third force that is less than the second force, and a fourth perimeter.

2. The dermaplaning blade-wiping pad system of claim 1 wherein the fourth perimeter of the release liner element extends outside the first perimeter of the blade-wiping pad by a length on at least the first side of the perimeter edge of the blade-wiping pad.

3. The dermaplaning blade-wiping pad system of claim 2 wherein the release liner element is configured with flexibility to permit bending the release liner element fourth bottom surface back upon itself under the blade-wiping pad where the fourth top surface of the release liner element touches the first perimeter of the blade-wiping pad on a perimeter edge side were the length of release liner element exists and wherein the length of release liner element is configured to permit a person with fingers to grasp the edge of the blade-wiping pad, remove the blade-wiping pad from the top surface of the release liner element and place the blade-wiping pad on the back outside surface of the first disposable medical glove to be adhered to the back outside surface of the first disposable medical glove.

4. The dermaplaning blade-wiping pad system of claim 3 wherein fingers to grasp the edge of the blade-wiping pad may be finger pads of fingers of a second disposable medical glove.

5. The dermaplaning blade-wiping pad system of claim 1 wherein the perimeter edge of the blade-wiping pad may have a shape from a group consisting of a square, a rectangle, a triangle, a circle, an oval.

6. The dermaplaning blade-wiping pad system of claim 1 wherein the first depth and second depth is at least 0.2 mm (8 mils) 0.1 mm (4 mils) and no more than 0.5 mm (20 mils), the second width and first cross-section are at least 0.5 mm (20 mils) and no more than 1.5 mm (60 mils), and the first thickness is at least 0.23 mm (9 mils) and no more than 3.0 mm (120 mils) to permit removal and capture of the skin debris collected on the blade during the dermaplaning procedure.

7. The dermaplaning blade-wiping pad system of claim 1 wherein the at least one collection of multiple parallel ridges between multiple parallel crevices in the top surface layer is one collection with all ridges parallel in the same direction and the collection of multiple parallel ridges and multiple parallel crevices is configured to be opposed to the direction of a wiping motion of the blade with skin debris by an angle consisting of more than 15 degrees, more than 30 degrees, more than 45 degrees, more than 60 degrees, and up to 90 degrees.

8. The dermaplaning blade-wiping pad system of claim 1 wherein the at least one collection of multiple parallel ridges between multiple parallel crevices in the top surface layer comprises two collections with half of ridges of one collection parallel in one direction with each ridge having a first width and a with each crevice having the first depth and the second width and half of ridges of the second collection parallel in another direction with ridge having a third width and each crevice having a third depth and a fourth width and positioned at an angle consisting of more than 15 degrees, more than 30 degrees, more than 45 degrees, more than 60 degrees, and up to 90 degrees to form a crosshatched pattern with similar parallelogram-shaped cavities each with an area formed by the second width and the fourth width and configured to remove and capture skin debris collected on the blade during the dermaplaning procedure when the blade is wiped on the blade-wiping pad.

9. The dermaplaning blade-wiping pad system of claim 8 wherein the second collection of multiple parallel ridges between multiple parallel crevices in the top surface layer is positioned at an angle consisting of approximately 90 degrees to form a crosshatched pattern with similar rectangular-shaped cavities each with an area formed by the second width and the fourth width and configured to remove and capture skin debris collected on the blade during the dermaplaning procedure when the blade is wiped on the blade-wiping pad.

10. The dermaplaning blade-wiping pad system of claim 1 wherein the multiple cavities in the top surface layer form a collection of similar patterns from a group consisting of circles, triangles, squares, rectangles, parallelograms, pentagons, hexagons, and irregular shapes to form cavities with an area configured to remove and capture skin debris collected on the blade during the dermaplaning procedure when the blade is wiped on the blade-wiping pad.

11. The dermaplaning blade-wiping pad system of claim 1 wherein the second force is less than the first force and the second force is configured to be more than a fourth force holding the third bottom surface of the pressure sensitive adhesive layer to the disposable medical glove digit pads during transfer of the blade-wiping pad from the release liner element to the back outside surface of the disposable medical glove.

12. The dermaplaning blade-wiping pad system of claim 1 wherein the fifth thickness of the release liner element is at least 0.1 mm (4 mils) and no more than 0.5 mm (20 mils).

13. The dermaplaning blade-wiping pad system of claim 1 wherein the contoured surface layer of the blade-wiping pad of claim 1 further comprises a color having a contrast between the skin debris and the blade-wiping pad to promote easy visibility of the skin debris.

14. The dermaplaning blade-wiping pad system of claim 13 wherein the color of the contoured surface layer is a dark color when the skin debris is more distinguishable against a dark color and a light color when the skin debris is more distinguishable against a light color than a dark color.

15. The dermaplaning blade-wiping pad system of claim 2 wherein the length between the first perimeter of the blade-wiping pad and the fourth perimeter of the release liner element is on the first side of the first perimeter edge of the blade-wiping pad and is from a group consisting of at least 1 mm (40 mils), at least 2 mm (80 mils), at least 3 mm (120 mils), at least 4 mm (160 mils), and at least 5 mm (200 mils).

16. The dermaplaning blade-wiping pad system of claim 2 wherein length between the first perimeter of the blade-wiping pad and the fourth perimeter of the release liner element is on the first side and at least the second side of the first perimeter edge of the blade-wiping pad and is from a group consisting of at least 1 mm (40 mils), at least 2 mm (80 mils), at least 3 mm (120 mils), at least 4 mm (160 mils), and at least 5 mm (200 mils).

17. The dermaplaning blade-wiping pad system of claim 1 wherein the contoured sheet element comprises a group consisting of a thermoplastic material and a fabric.

18. The dermaplaning blade-wiping pad system of claim 1 wherein the pressure sensitive adhesive layer comprises a pressure sensitive adhesive formed from a group consisting of acrylic, silicone, rubber, silicone-rubber, and modified epoxy.

19. A method of using a dermaplaning blade-wiping pad system for removing and capturing human skin debris collected on a dermaplaning blade during a dermaplaning procedure by a skin care person comprising the steps of,
providing at least a first disposable medical glove comprising an inside surface and an outside surface with a back outside surface opposite a palm outside surface and fingers with finger pads on the hand of the skin care person that is to hold the skin taut during dermaplaning;
providing the dermaplaning blade-wiping pad system for removing and capturing human skin debris collected on a blade during a dermaplaning procedure by a skin care person comprising,
a blade-wiping pad having a first perimeter, a perimeter edge with a first side and at least a second side, and comprising
a contoured sheet element comprising an area, a first thickness, a second perimeter, a bottom surface layer comprising a second thickness, a first top surface and a first bottom surface, and a top surface layer comprising a third thickness, a second top surface having a multitude of contours and a second bottom surface attached to the first top surface of the bottom surface layer to form hollow open chambers configured to remove and capture the skin debris collected on the blade during a dermaplaning procedure wherein the multitude of contours comprises at least one of a group consisting of (1) at least one collection of multiple parallel ridges, each ridge comprising a first height and at least a first width between multiple parallel crevices, each crevice comprising a first depth, and at least a second width and (2) multiple cavities in the top surface layer with a second depth and a cross-section wherein the first height is the same as the first depth; and
a pressure sensitive adhesive layer comprising a fourth thickness, a third top surface affixed to the first bottom surface of the bottom surface layer of the contoured sheet element with a first force, a third bottom surface configured to releasably adhere with a second force to a back outer surface of a first disposable medical glove during the dermaplaning procedure with the first force greater than the second force, and a third perimeter equal to the second perimeter of the contoured sheet element to form the first perimeter of the blade-wiping pad so the entire second bottom surface of the top surface layer is in contact with the third top surface of the pressure sensitive layer; and
a release liner element comprising a fourth bottom surface, a fifth thickness, a fourth top surface releasably adhered to the third bottom surface of the pressure sensitive adhesive layer with a third force that is less than the second force, and a fourth perimeter;
providing the dermaplaning blade to remove skin debris from skin during dermaplaning;
removing the blade-wiping pad from the release liner element and attaching the blade-wiping pad to the back outside surface of the first disposable medical glove;
removing skin debris from skin held taut during dermaplaning and transferring the skin debris onto the dermaplaning blade without needing to move eyes and attention from the skin that is being dermaplaned;
wiping the dermaplaning blade on the second top surface of the blade-wiping pad to remove and capture the skin debris from the blade; and
repeating the last two steps until the dermaplaning procedure is complete.

20. The method of claim 19 further comprising the steps of:
removing the first disposable medical glove with the blade-wiping pad with skin debris by grasping the wrist area and pulling the first disposable medical glove off the hand while allowing the first disposable medical glove to become inside out and thereby confining the blade-wiping pad with skin debris in a sanitary manner, and
disposing of the inside out first disposable medical glove containing the blade-wiping pad with skin debris.

21. The method of claim 19 wherein the blade-wiping pad may be removed from the release liner element with a second disposable medical glove worn by the skin care person on the dominant hand.
